(19) United States
(12) Patent Application Publication
(10) Pub. No.: US 2002/0128576 A1
(43) Pub. Date: Sep. 12, 2002

(54) Back rehab exercise bench

(76) Inventor: Billy Jack Mayes, Brevard, NC (US)

Correspondence Address: Billy Jack Mayes, 95 Deerwooded Lane, Brevard, NC, 28712, US

(21) Appl. No.: 09/802,012

(22) Filed: Mar. 9, 2001

(51) Int. Cl. A61F 5/00

(52) U.S. Cl. 602/33; 602/32

(57) ABSTRACT

An exercise table and exercise routine usefull in the treatment of low back pain is disclosed. The table design permits a user to apply low levels of traction to the lower back by pushing lightly on adjustable arm support posts. The table design permits the user to have complete control of the level of traction applied to the lower back. An exercise routine uses the methodology of relaxing the user and applying very low levels of traction to the lower back. The low level of traction is alternated every few seconds and utilizes deep breaths to keep the user in a relaxed condition.

### FIELD OF INVENTION

The present invention generally relates to the therapeutic treatment of the back and more particularly to the treatment of the lower back.

### BACKGROUND OF INVENTION

Back troubles affect millions of people each year. Pain in the lower lumbar area of the back is the most common back difficulty. In response to this frequent problem, numerous treatments have been developed, recommended, and practiced. These treatments involve exercise therapy, specialized equipment, and even surgery to relieve the discomfort associated with lower back trauma.

Lower back therapy usually involves procedures that stretch or elongate the spine and decompress the vertebrae that contribute to back pain. Common procedures use various forms of traction using weights and even the individuals own body weight to stretch the back and provide pain relief.

Procedures that use the patient's weight and suspend the person in an inverted, head-down position have been and are currently being used to stretch and decompress the back. Using gravity and the individual's body weight to stretch the back in this fashion has several disadvantages. In order for the back to properly decompress the person must be relaxed and hanging in an inverted state is not a relaxed position due to fear and to discomfort from being inverted. Supporting body weight with arms or elbows or being suspended in an inverted position may give a feeling of relief initially but after a short time muscles tighten and reduce and even eliminate the spinal relaxation required to alleviate the pain. It is very difficult to remain in a relaxed condition while supporting even a portion of your body weight. When muscles become tense, the spine is prevented from decompressing and the relief is limited.

Therapeutic tables have been utilized for applying traction to the users lumbar region and achieving pain relief. These procedures involve the patient lying horizontal, either face down or face up on the table and mechanical traction is applied to stretch or decompress the affected area of the back. Traction is usually applied to the back using weights and pulleys. More recently, tables have been proposed that have movable sections where the individual is held fixed and traction is obtained by separation of the table sections. The separation of the table sections and the application of traction has been accomplished using cables and a winch, hydraulic cylinders, and a motor driven device.

Although various therapeutic traction tables are disclosed, few provide the user complete control of the amount of traction and stretch applied to their back and none provided specific procedures and methodology to utilize very low levels of traction to relieve back pain. The relief of back pain can best be accomplished when the back and the body in general are in a relaxed condition. The muscles that surround and protect the spine must be relaxed to permit the spine to be stretched and decompressed. Traction without relaxation will not successfully relieve the pressures that contribute to back pain.

### BRIEF SUMMARY OF THE INVENTION

According to this invention, an exercise table is described that permits a user to apply very low levels of traction to the lower back. A user driven routine is also described that enables the body, especially the back, to relax allowing the lower spine to be efficiently and effectively stretched and decompressed. The exercise table consists of a frame that contains two parts, a lower portion that is fixed and an upper portion that is moveable. The movement of the sections of the table does not apply traction or stretching force to the user's back. The movement of the table sections compensates for stretch in the harness used to attach the feet to foot brackets and for the elongation of the back as the treatment progresses. The force needed to stretch and decompress the back is generated by the user lightly pushing against padded posts that are attached to the upper frame. Each user determines and controls the level of traction applied to the back. As treatment progresses and the back is stretched, the user adjusts the table sections to eliminate slack and maintain the position of the padded posts such that light pressure by the user's elbows retains traction on the back. The traction is not constant but is alternated every few seconds using deep breaths to keep the body in a relaxed condition.

The table design and the harnesses used permits the feet and arms to be easily disconnected, allowing specific body movements that assist in treating lower lumbar pain. The procedural methodology that is used by the patient while on the table is very important for successful reduction and even elimination of back pain. Using the table and applying traction to the lower back without using the methodology to achieve relaxation and muscle stretch, will result in a less successful outcome. The design of the table is important in that it provides a means for the user to apply the proper amount of traction to stretch and decompress the back. The procedures and methods described in this invention are, however, equally important for relieving lower back pain.

The table described in this invention is designed to permit the user to control the traction applied to the back and permit a slow extension of the lower back. The table also permits the user to move and stretch out of plane with one leg while retaining the other leg in a stretched condition. The amount of traction applied to the back is very low and is easily controlled by the user. This control and the procedures described in this invention permit the user to relax and allow the low traction forces to gently stretch the back. The relaxation of the muscles in the back is important and significantly impacts the success of this form of treatment.

The separation of the sections of the exercise table is accomplished using an electric motor driving a threaded rod. The user controls the movement of the table sections with a hand held push button control module. The table sections can also be moved using hydraulic methods but the use of an electric drive easily permit the incorporation of safety protocols in the design.

### BRIEF DESCRIPTION OF DRAWINGS

FIG. 1 is a top view of the table that incorporates the principles of the invention.

FIG. 2 is a side view of the table showing the major components of the design.

FIG. 3 is an isometric view of the table showing the major components of the design.

### DETAILED DESCRIPTION OF THE INVENTION

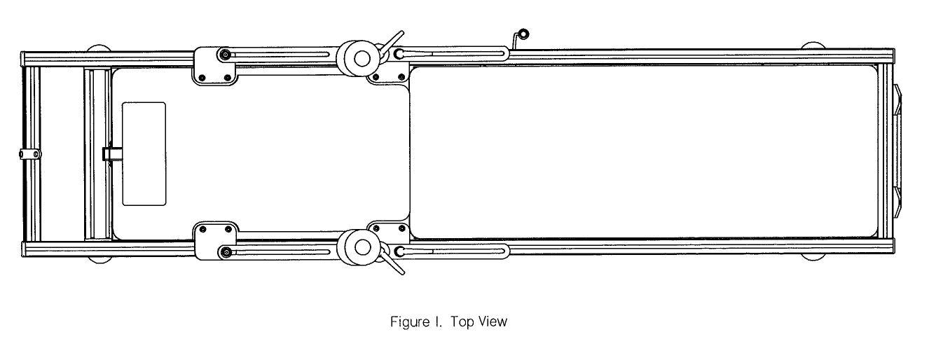

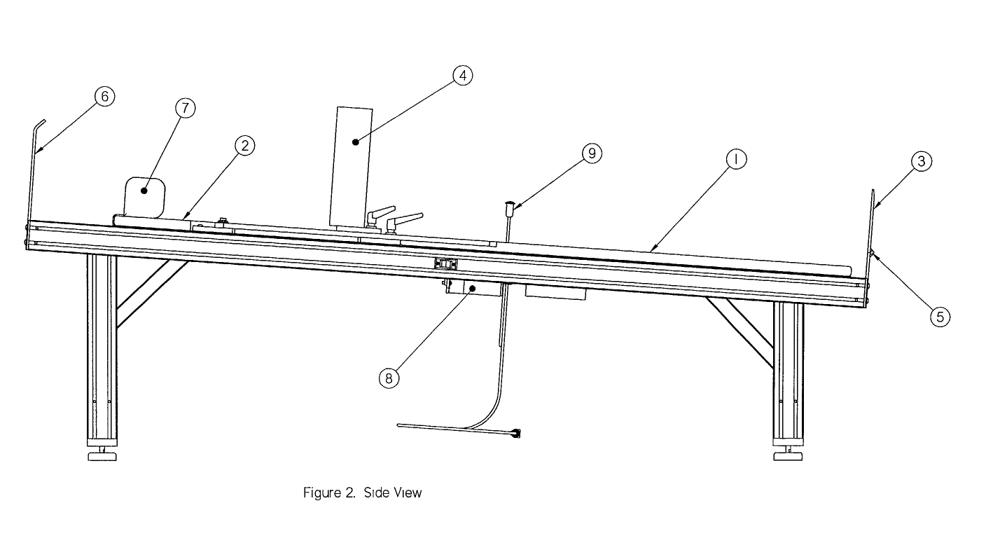

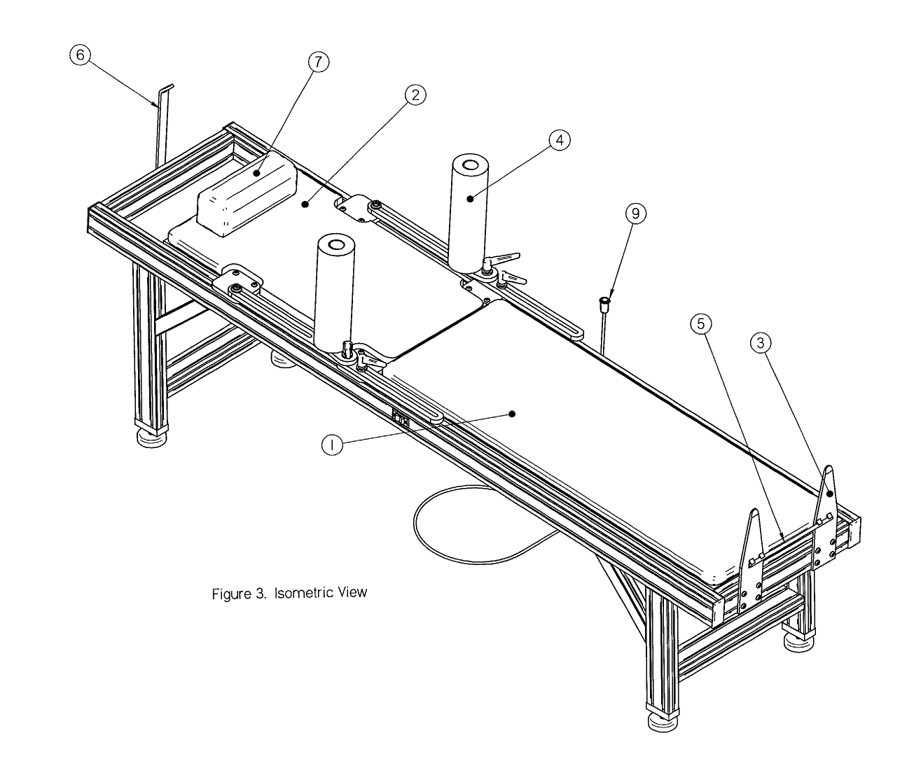

The exercise table described in this invention is shown in FIGS. 1, 2, and 3. The table frame is 85 inches long and 20 inches wide. The frame legs are 24 inches high at the head end of the table and 18 inches high at the foot end. This 5-degree incline, with the head being higher than the feet, is more comfortable for the user, assists in circulation, and aids in relaxation. Referring to FIGS. 2 and 3, the padded table top is in two parts, a lower 49 inch long section 1 that is fixed and an upper 36 inch long section 2 that is moveable. The table is provided with two flat foot pegs 3 at the lower end to attach a harness that is placed on each of the user's feet. The harnesses may be attached or unattached by sliding the harness over the flat foot pegs. The table contains arm posts 4 that are attached to the moveable portion of the table. The arm posts are 12 inches long and are covered with a I inch thickness of foam resulting in a total diameter of 3.5 inches. The arm posts support the user's arms. The arm posts are adjustable to accommodate individuals from 5 feet 0 inches to 6 feet 8 inches in height and with chest widths ranging from 12 inches to 18 inches. For users having knee or hip problems, such as knee or hip replacements, a pelvic harness is provided which is attached to the connectors 5 positioned on the frame of the table. The table also contains a support 6 to attach a neck harness and a cushion 7 to support the user's head.

The upper and lower sections of the table are separated by means of an electric motor 8 that drives a threaded rod. The activation of the motor and control of the amount of separation of the table sections is achieved by the user operating buttons on a control module 9.

The use of the table begins with the two sections of the table closed together and the arm posts adjusted in height and width to generally fit under the user's arms. A simple harness is attached to the user's feet. The user lies on the table face-up and the feet are attached to the lower, fixed section of the table by sliding the harness on each foot over the appropriate foot peg. For users with knee or hip problems a pelvic harness is provided which is attached to each side of the frame. The user's arms are placed over the arm posts and the arm posts are readjusted to be comfortable for the user and to remove any slack in the harness or position of the body on the table. If the user is not snug, the table can be extended by pushing the control button on the hand held controller to expand the table sections. With the table snug, the user is positioned with the back of their arms or triceps on the arm posts, the arms are bent, and their hands are placed on their forehead. This has been found to be a very comfortable position. While snuggly fitted in the table, the user can very simply apply traction to the lower back by pushing the back of the arms against the arm posts. A male will normally develop between 10 to 20 pounds of downward pressure by pushing lightly on the arm posts while a female will generate from 5 to 10 pounds of pressure by pushing on the arm posts.

The table is designed to permit the user to apply low amounts of traction to stretch the lower back and relieve pain. In order for the back to respond to the low levels of traction, it is extremely important for the user to be relaxed. The treatment routine generally includes several steps to initially relax the user and begin to elongate the back followed by steps to further stretch the back and to build the muscles in the lower back. In order to relax, it is important for the user to be comfortable on the table and to understand that they are in control of the traction applied to the back. It is also important for the user to understand that very low levels of traction are required to stretch the lower back. The initial treatment steps permit the user to become comfortable on the table while lightly stretching the lower back. The additional treatment sets elongate the back and provide exercises that strengthen the muscles in the lower back.

A treatment procedure that has been successfully employed in relieving lower back pain is described below. It is recognized that other procedures may also be used with the table but the treatment described below has proven successful on a variety of users. The treatment involves three sets of traction exercises, which are given below:

Set 1.

1. The user pushes against the arm posts, takes a deep breath and holds for 10 seconds.

The stomach is held tight and the lower back is pushed against the table. The breath is slowly let out and the user relaxes.

2. The user then relaxes the back by slowly flexing the legs and buttocks almost in a kneading action.

3. The user pushes against the arm posts, takes a deep breath, holds for 5 seconds, and then relaxes. This is repeated 5 times.

4. The user relaxes and moves the legs and buttocks as in step 2.

5. Then a deep breath is taken, pressure is applied to the arm posts and this is held for 10 seconds while the toes are moved.

6. Steps 1, 2, 3, 4, and 5 are then repeated.

7. At this point, it may be necessary to expand the table sections to maintain a snug fit between the user and the table. It is recommended that the table not be extended more than 1.0 inches at any one time.

Set 2

8. Step 1 and 2 above are repeated.

9. The user takes a deep breath, pressure is applied to the arm posts, and with the stomach held tight the pelvis is pushed against the table. This is held for 10 seconds.

10. The user then relaxes and Step 2 is repeated.

11. The user takes a deep breath, pushes on the arm posts, and again pushes the pelvis against the table. This is held for 5 seconds and then relaxed. This is repeated 5 times.

12. The user relaxes and Step 2 is repeated.

13. The user slips the harness of the right leg off the right foot peg and slips the left arm off the left arm post. The right leg is bent and knee is grabbed and held by both hands. While pushing down on the left arm post and stretching the left leg, the right leg is pulled upward into the body, stretching the lower back. This is held for 10 seconds. The right leg is now pulled over to the left side as far as possible and while pushing on the left arm post to apply light traction to the left leg, the knee is pulled using both hands stretching the lower back. This is held for 20 seconds.

14. Step 13 is repeated using the alternate leg.

15. The user relaxes and Step 2 is repeated.

16. The table may be adjusted as required to expand the sections and keep the body snug.

Set 3

17. Repeat Steps 8 through 16.

18. The user applies light traction by pushing on the arm posts and holds for 10 deep breaths.

19. The user totally relaxes for 2-3 minutes while lying on the table.

A user can complete the above routine in 12 to 15 minutes. The routine seems to involve many steps but the steps require only seconds to complete and on inspection, the steps are repetitious in nature.

The table described in FIGS. 1, 2, and 3 permits an individual with lower back pain to easily apply low levels of traction to the lower back. The table is able to adjust to compensate for elongation of the back or to take up slack in the harness. The level of traction is totally controlled by the user pushing on arm support posts. An exercise routine enables the user to relax and stretches and decompresses the back by alternating traction and deep breaths every few seconds. The exercise routine includes steps that strengthen the muscles of the lower back. Other routines may be described or developed but the routine described herein has proven to successfully relieve lower back pain in a large number of users.

What is claimed is:

1. A therapeutic table usefull in the treatment of low back pain, the table comprising:
a frame sufficiently long and wide to support a patient from 5 feet 0 inches to 6 feet 8 inches in height;
a table top having a fixed lower (foot end) section and a moveable upper section;
an electric motor that operates a threaded rod that moves the upper table section relative to the fixed lower section;
a push button control module that is operated by the patient controls the electric motor and determines the separation of the table sections;
a means to restrain the lower body to the fixed frame using removable harnesses attached to the feet or by means of a pelvic belt attached to the fixed frame;
a pair of adjustable arm support posts that are attached to the upper movable section of the table that permit the user to stretch and decompress the lower back by applying light pressure on the arm support posts;

2. The table according to claim 1, wherein the upper section is movable with respect to the lower section serves to maintain the patient's back in a snug condition.

3. The table according to claim 1, wherein the arm support posts are positioned to permit the user to apply traction to the lower back by lightly pushing on the arm posts.

4. The table design permits the user to totally control the traction applied to the lower back.

5. An exercise routine that uses a methodology of applying low levels of traction to the user's lower back, the low traction force alternating every few seconds with deep breaths to maintain the user in a relaxed condition.

6. The routine according to claim 5, wherein exercises are used to strengthen the lower back muscles while the back is under traction.
